(19) United States
(12) Patent Application Publication
(10) Pub. No.: US 2025/0239333 A1
(43) Pub. Date: Jul. 24, 2025

(54) SEARCHING A CHEMICAL STRUCTURE DATABASE BASED ON CENTROIDS

(71) Applicant: Camelot UK Bidco Limited, London (GB)

(72) Inventor: Ryan BRADY, Denver, CO (US)

(21) Appl. No.: 18/421,694

(22) Filed: Jan. 24, 2024

(51) Int. Cl. G16C 20/40; G16C 20/70

(52) U.S. Cl. CPC G16C 20/40; G16C 20/70

(57) ABSTRACT

System, methods, apparatuses, and computer program products are disclosed for searching a chemical structure database based on centroids. A query is executed against a chemical structure database by first vectorizing a molecular representation associated with the query into a query feature vector. The query feature vector is compared to centroid feature vectors to determine a centroid feature vector associated with a chemical structure representation that satisfies a similarity condition with the query feature vector. A first subset of the chemical structure database that includes chemical structures of the chemical structure database associated with the determined centroid feature vector is searched to determine whether the molecular representation associated with the query is present in the first subset of the chemical structure database.

### BACKGROUND

Searching large chemical structure databases presents numerous challenges due to the vastness and complexity of the search space. Due to their size, rapid and accurate searches of chemical structure databases demand enormous computational power. As the size of the chemical structure database increases, search times may escalate, impacting user experience. The computational costs and time required to determine whether a compound is novel may be significant and even hinder the research process.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

System, methods, apparatuses, and computer program products are disclosed for searching a chemical structure database based on centroids. A query is executed against a chemical structure database by first vectorizing a molecular representation associated with the query into a query feature vector. The query feature vector is compared to centroid feature vectors to determine a centroid feature vector associated with a chemical structure representation (e.g., a Markush structure) that satisfies a similarity condition with the query feature vector. A first subset of the chemical structure database that includes chemical structures of the chemical structure database associated with the determined centroid feature vector is searched to determine whether the molecular representation associated with the query is present in the first subset of the chemical structure database.

Further features and advantages of the embodiments, as well as the structure and operation of various embodiments, are described in detail below with reference to the accompanying drawings. It is noted that the claimed subject matter is not limited to the specific embodiments described herein. Such embodiments are presented herein for illustrative purposes only. Additional embodiments will be apparent to persons skilled in the relevant art(s) based on the teachings contained herein.

### BRIEF DESCRIPTION OF THE DRAWINGS

The accompanying drawings, which are incorporated herein and form a part of the specification, illustrate embodiments of the present application and, together with the description, further serve to explain the principles of the embodiments and to enable a person skilled in the pertinent art to make and use the embodiments. Dashed portions of the drawings may represent optional steps and/or elements.

FIG. 1 shows a block diagram of an example system for searching a chemical structure database based on centroids, in accordance with an embodiment.

FIG. 2 shows a block diagram of an example system for indexing a chemical structure database based on centroids of a group of chemical structures, in accordance with an embodiment.

FIG. 3 depicts a flowchart of a method for searching a first subset of a chemical structure database based on centroids, in accordance with an embodiment.

FIG. 4 depicts a flowchart of a method for searching a second subset of a chemical structure database based on centroids, in accordance with an embodiment.

FIG. 5 depicts a flowchart of a method for searching a third subset of a chemical structure database based on centroids, in accordance with an embodiment.

FIG. 6 depicts a flowchart of a method for indexing a chemical structure database based on a centroid of a group of structures, in accordance with an embodiment.

FIG. 7 depicts a flowchart of a method for indexing a chemical structure database based on a centroid of a group of chemical structures, in accordance with an embodiment.

FIG. 8 shows a block diagram of an example computer system in which embodiments may be implemented.

The features and advantages of the embodiments described herein will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION

### I. Introduction

The following detailed description discloses numerous example embodiments. The scope of the present patent application is not limited to the disclosed embodiments, but also encompasses combinations of the disclosed embodiments, as well as modifications to the disclosed embodiments. It is noted that any section/subsection headings provided herein are not intended to be limiting. Embodiments are described throughout this document, and any type of embodiment may be included under any section/subsection. Furthermore, embodiments disclosed in any section/subsection may be combined with any other embodiments described in the same section/subsection and/or a different section/subsection in any manner.

### II. Example Embodiments

Scientists researching chemical compounds may need to determine whether a chemical compound is novel. For instance, scientists in pharmaceutical research may need to determine whether a chemical compound has already been discovered, disclosed, and/or patented in order to focus their research on commercially viable chemical compounds. In embodiments, this may require searching for a chemical compound in one or more chemical structure databases that encompass a vast majority, or all, of the known chemical compounds. Thoroughly searching such a large search space requires vast amounts of computational resources and/or time, resulting in higher research costs and/or research delays.

Embodiments described herein are directed to indexing and/or searching of chemical structure databases based on centroids associated with chemical structure representations, such as, but not limited to, Markush structures. Markush structures are molecular representations used to describe a group or family of chemical compounds within a single structure. These structures are defined by a core scaffold with variable substituents or R-groups, allowing for the inclusion of numerous related compounds that share a similar core backbone but differ in specific functional groups or side chains. Embodiments disclosed herein leverage the similarities between compounds that satisfy a Markush structure to focus the search space. It is noted that although Markush structures are often referenced herein, embodiments are applicable to chemical structure representations other than Markush structures, as would be known to persons skilled in the relevant art(s). For instance, chemical structure representations may, in embodiments, include a chemical structure representation comprising a core molecular property, such as, but not limited to, a structural element, a functional element, a molecular property, and/or the like, wherein the chemical structures of the first group of chemical structures share the core molecular property.

Indexing a chemical structure database based on centroids associated with chemical structure representations, such as, but not limited to, Markush structures, may, in embodiments, include determining a centroid feature vector that is representative of compounds that satisfy the chemical structure representation, and then associating the centroid feature vector with the chemical structures in the chemical structure database that satisfy the chemical structure representation. For instance, a centroid feature vector may be determined for a chemical structure representation by averaging each dimension of a plurality of individual feature vectors that are each representative of a compound that satisfies the chemical structure representation. In embodiments, the centroid feature vector may be iteratively determined based on a set of randomly generated compounds that satisfy the chemical structure representation. For instance, given a Markush structure, a random compound may be generated by randomly selecting a permissible substituent for each R-group in the Markush structure. Once a plurality of compounds that satisfy the Markush structure are randomly generated, an initial centroid feature vector may be determined based on individual feature vectors associated with each of the plurality of randomly generated compounds. In subsequent iterations, the centroid feature vector may, in embodiments, be recalculated based on additional randomly generated compounds that satisfy the Markush structure. In embodiments, this process may continue until the difference in centroid feature vectors between temporally adjacent iterations satisfies a convergence condition, such as, but not limited to, a percentage difference threshold, an absolute difference threshold, a distance threshold, and/or the like. Chemical structure database may be indexed based on the determined centroid feature vector by associating chemical structures in the chemical structure database that satisfy the chemical structure representation with the determined centroid feature vector.

In embodiments, a chemical structure database may be indexed based on a plurality of clusters of chemical structures that satisfy a chemical structure representation, such as a Markush structure. For instance, when a chemical structure representation encompasses a large number of chemical structures, there may be greater variance between the chemical structures that satisfy the chemical structure representation. In embodiments, greater variance between the chemical structures that satisfy the chemical structure representation may result in distinct groupings or clusters of chemical structures that have a high level of similarity. The similarity of chemical structures within these clusters may, in embodiments, be leveraged to improve searching by calculating a centroid feature vector for each of these clusters and indexing the chemical structure database based on the determined centroid feature vectors. In embodiments, centroid feature vectors may be determined for clusters within chemical structures that satisfy a chemical structure representation in a manner similar to determining a centroid feature vector for chemical structures that satisfy a chemical structure representation, as described above.

Searching for a molecular representation in a chemical structure database based on centroids associated with chemical structure representations, such as, but not limited to, Markush structures may, in embodiments, include determining the chemical structure representations having the highest similarity to the molecular representation, and searching for the molecular representation in a subset of chemical structure database that includes the chemical structures related to the chemical structure representations having the highest similarities to the molecular representation. For instance, similar chemical structure representations may be determined for a molecular representation of a queried compound by generating a query feature vector representative of the molecular representation and comparing the query feature vector to centroid feature vectors associated with a plurality of candidate chemical structure representations. In embodiments, a search space may be determined as a subset of the chemical structure database associated with centroid feature vectors having similarities to the query feature vector that satisfy a similarity condition, such as but not limited to, a similarity threshold, a difference threshold, a distance threshold, and/or the like. For instance, the search for the molecular representation of the queried compound may, in embodiments, be limited, at least initially, to the chemical structures associated with centroid feature vectors having similarities to the query feature vector that satisfy a similarity condition. Limiting the search space based on centroids associated with chemical structure representations having a high similarity to the molecular representation may result in improved search times and/or reduced computational costs.

In embodiments, searching for a molecular representation in a chemical structure database based on centroids associated with chemical structure representations may be performed in a plurality of phases. For instance, a first phase may be performed by determining a first set of centroid feature vectors that satisfy a first similarity condition (e.g., a first similarity threshold) with the query feature vector, and searching for the molecular representation in a first subset of the chemical structure database that includes the chemical structures associated with the first set of centroid feature vectors. If the first phase results in no positive matches, a second phase may be performed based on a second similarity condition that may, in embodiments, result in a larger search space. For instance, a second phase may be performed by determining a second set of centroid feature vectors that satisfy a second similarity condition (e.g., a second similarity threshold) with the query feature vector, and searching for the molecular representation in a second subset of the chemical structure database that includes the chemical structures associated with the second set of centroid feature vectors. Since the first subset of the chemical structure database has already been searched in the first phase, the second subset of the chemical structure database may, in embodiments, exclude the first subset of the chemical structure database. If the second phase results in no positive matches, additional phases may, in embodiments, be performed based on additional similarity conditions that may each result in a larger search space. In embodiments, one or more of the similarity conditions may be manually, automatically, and/or semi-automatically determined based on input from one or more of: a user, a customer, a subject matter expert, historical data, and/or the like. In embodiments, a final phase may be performed by searching for the molecular representation in the portions of the chemical structure database not included in the previous phases of the search.

Performing the search in phases may improve search efficiency for a large number of searches without any impact to the accuracy and/or thoroughness of the search. For instance, an initial phase may be performed very quickly, but may result in a higher probability of a false negative result. Subsequent phases of the search may increase search times, but also decrease the probability of a false negative result. The final phase of the search may be performed to exclude the possibility of a false negative result.

Embodiments disclosed herein may include an interface, such as, but not limited to, a user interface (UI), a graphical user interface (GUI), a command-line interface (CLI), an application programming interface (API), and/or the like, to facilitate indexing and/or searching of chemical structure databases based on centroids associated with chemical structure representations, such as, but not limited to, Markush structures. The interface may, in embodiments, enable input of a molecular representation of a queried compound. The molecular representation may, in embodiments, include, but is not limited to, a machine-readable molecular representation, a Simplified Molecular Input Line Entry System (SMILES) string, a connection table, an atom connectivity matrix, a molfile, a chemical table file, an RGFile, and/or a machine-learning representation. In embodiments, the molecular representation may be provided through various ways, such as, but not limited to, by uploading a file containing the molecular representation, by inputting the molecular representation as text, by drawing a graphical molecular representation, by verbally dictating or describing the molecular representation, and/or the like. In embodiments, the interface may include elements to enable configuration and/or customization of search parameters, such as, but not limited to, similarity conditions, similarity thresholds, number of search phases, maximum search time, and/or the like. In embodiments, the interface may include elements to direct the flow of the search, such as, but not limited to, starting a new search, scheduling a search, stopping a search, selecting chemical structure representations of interest, and/or the like.

These and further embodiments are disclosed herein that enable the functionality described above and additional functionality. Such embodiments are described in further detail as follows.

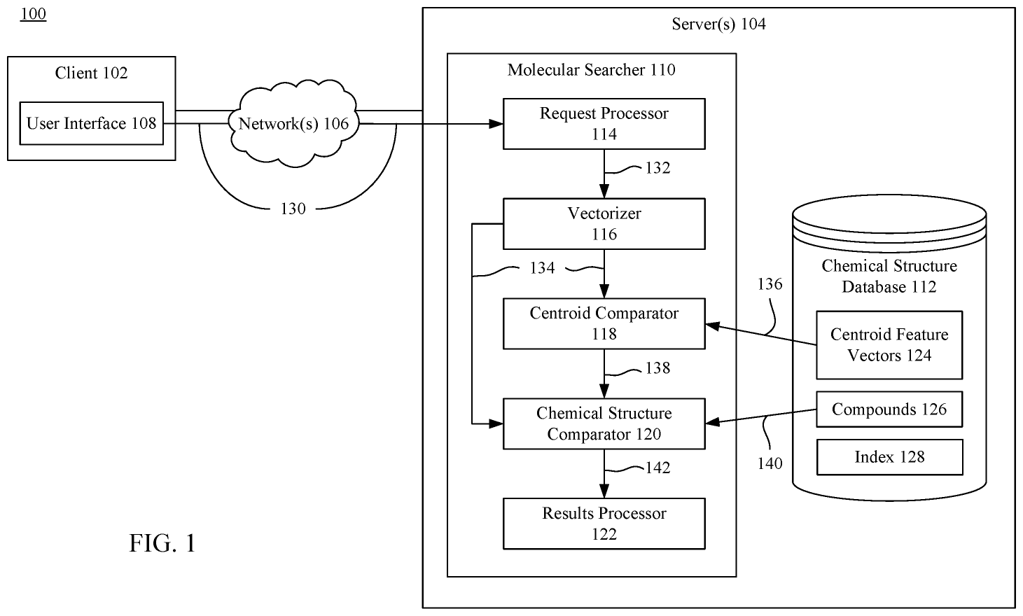

For instance, FIG. 1 shows a block diagram of an example system 100 for searching a first subset of a chemical structure database based on centroids, in accordance with an embodiment. As shown in FIG. 1, system 100 includes a client 102 and one or more servers 104, which are communicatively coupled to each other via one or more networks 106. Furthermore, client 102 includes a user interface (UI) 108, and server(s) 104 includes a molecular searcher 110 and a chemical structure database 112. As shown in FIG. 1, molecular searcher 110 further includes a request processor 114, a vectorizer 116, a centroid comparator 118, a chemical structure comparator 120, and a results processor 122. Moreover, chemical structure database 112 further includes centroid feature vectors 124, compounds 126, and an index 128. System 100 is described in further detail as follows.

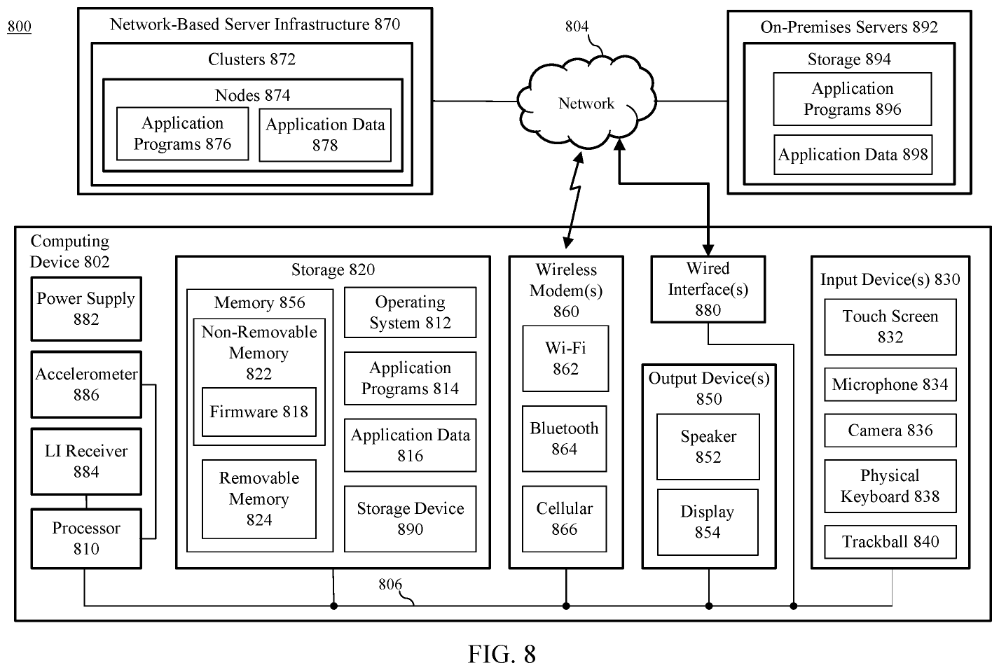

Network(s) 106 may comprise one or more networks such as local area networks (LANs), wide area networks (WANs), enterprise networks, the Internet, etc., and may include one or more wired and/or wireless portions. Various example implementations of network(s) 106 are described below in reference to FIG. 8 (e.g., network 804, and/or components thereof).

Client 102 may comprise any type of stationary or mobile processing device, including, but not limited to, a desktop computer, a server, a mobile or handheld device (e.g., a tablet, a personal data assistant (PDA), a smart phone, a laptop, etc.), etc. As shown in FIG. 1, client 102 includes UI 108 that enables transmission of a query 130 to server(s) 104. Various example implementations of client 102 are described below in reference to FIG. 8 (e.g., computing device 802, and/or components thereof).

In embodiments, UI 108 may comprise various interfaces, such as, but not limited to, a user interface (UI), a graphical user interface (GUI), a command-line interface (CLI), an application programming interface (API), and/or the like, to facilitate searching of chemical structure databases based on centroids associated with chemical structure representations, such as, but not limited to, Markush structures. UI 108 may, in embodiments, enable input, as part of query 130, a molecular representation of a queried compound, such as, but not limited to, a machine-readable molecular representation, a Simplified Molecular Input Line Entry System (SMILES) string, a connection table, an atom connectivity matrix, a molfile, a chemical table file, an RGFile, and/or a machine-learning representation. In embodiments, UI 108 may enable the molecular representation to be input through various ways, such as, but not limited to, by uploading a file containing the molecular representation, by inputting the molecular representation as text, by drawing a graphical molecular representation, by verbally dictating or describing the molecular representation, and/or the like. In embodiments, UI 108 may include elements to enable configuration and/or customization of search parameters, such as, but not limited to, similarity conditions, similarity thresholds, number of search phases, maximum search time, whether an exact match is required, and/or the like. In embodiments, UI 108 may include elements to direct the flow of the search, such as, but not limited to, starting a new search, scheduling a search, stopping a search, selecting chemical structure representations of interest, and/or the like. Various example implementations of UI 108 are described below in reference to FIG. 8 (e.g., application 814, input device(s) 830, output device(s) 850, and/or components thereof).

Server(s) 104 may include physical servers, virtual servers, and/or a network-accessible server set (e.g., a cloud-based environment or platform). In an embodiment, the underlying resources of server(s) 104 may be co-located (e.g., housed in one or more nearby buildings with associated components such as backup power supplies, redundant data communications, environmental controls, etc.) to form a datacenter, may be distributed across different regions, and/or may be arranged in other manners. In accordance with an embodiment, server(s) 104 comprises part of a cloud computing platform. Various example implementations of server(s) 104 are described below in reference to FIG. 8 (e.g., network-based server infrastructure 870, on-premises servers 892, and/or components thereof).

Molecular searcher 110 may be configured to receive query 130 from client 102 via network(s) 106 and execute query 130 against chemical structure database 112 based on centroid feature vectors 124 associated with chemical structure representations, such as, but not limited to, Markush structures. In embodiments, molecular searcher 110 may be configured to provide results from executing query 130 to client 102 for output via UI 108.

Chemical structure database 112 may include one or more databases configured to store centroid feature vectors 124 associated with chemical structure representations, such as, but not limited to, Markush structures, compounds 126, and an index 128 that associates compounds 126 to one or more centroid feature vectors 124.

Request processor 114 is configured to receive query 130 and extract a molecular representation 132 from query 130. In embodiments, molecular representation 132 may include, but is not limited to, a machine-readable molecular representation, a Simplified Molecular Input Line Entry System (SMILES) string, a connection table, an atom connectivity matrix, a molfile, a chemical table file, an RGFile, and/or a machine-learning representation. In embodiments, query 130 may further include search parameters, such as, but not limited to, similarity conditions, similarity thresholds, number of search phases, maximum search time, whether an exact match is required, and/or the like. Request processor 114 is further configured to provide molecular representation 132 to vectorizer 116.

Vectorizer 116 is configured to receive molecular representation 132 from request processor 114, and generate a query feature vector 134 that is representative of molecular representation 132. In embodiments, query feature vector 134 is a numerical vector that captures various molecular properties of molecular representation 132. For instance, vectorizer 116 may, in embodiments, encode structural information, such as, but not limited to, atom types, bond types, and/or their spatial arrangements, into a numerical vector. In embodiments, vectorizer 116 may encode additional molecular properties into query feature vector 134, including, but not limited to, patterns of atoms and/or bonds, molecular weight, solubility, electronegativity, molecular conformations, spatial arrangements, and/or the like. In embodiments, vectorizer 116 may employ one or more hash functions to convert molecular representation 132 into query feature vector 134. In embodiments, vectorizer 116 may employ a machine learning (ML) encoder that is trained on a large set of chemical structures. For instance, vectorizer 116 may employ an ML encoder to transform molecular representation 132 into a condensed vector representation, such as query feature vector 134. In embodiments, an ML encoder may be trained by iteratively optimizing parameters to learn meaningful representations of input molecular representations by minimizing, over multiple iterations, a loss function that quantifies the difference between a generated representation and a target representation. In embodiments, the loss function may be based on whether or not molecular representations satisfy a common chemical structure representations, such as, but not limited to, a Markush structure. Vectorizer 116 may be configured to provide query feature vector 134 to centroid comparator 118 and/or chemical structure comparator 120.

Centroid comparator 118 is configured to determine a set of centroid feature vectors 124 that satisfy a similarity condition with query feature vector 134. For instance, centroid comparator 118 may be configured to receive query feature vector 134 from vectorizer 116 and one or more centroid feature vectors 136 from chemical structure database 122, and determine a centroid set 138 of centroid feature vectors with a similarity with query feature vector 134 that satisfy a predetermined similarity condition, such as, but not limited to, a similarity threshold, a distance threshold, a difference threshold, and/or the like. In embodiments, centroid feature vector(s) 136 may include some or all of centroid feature vectors 124. For instance, in embodiments, centroid comparator 118 may receive, as centroid feature vector(s) 136, centroid feature vectors 124 associated with chemical structure representations that satisfy one or more filtering conditions, such as, but not limited to, chemical structure representations that include a particular chemical element and/or element group, chemical structure representations that do not include a particular chemical element and/or element group, and/or the like.

In embodiments, centroid comparator 118 may determine a difference and/or distance between query feature vector 134 and centroid feature vector(s) 136, and determine whether the difference and/or distance satisfies a predetermined relationship with a similarity threshold. Various functions may be employed to determine a difference and/or distance between query feature vector 132 and centroid feature vector(s) 136, including, but not limited to, Euclidean distance, Manhattan distance, cosine similarity, and/or the like. Euclidean distance measures the straight-line distance between two points in space, considering each dimension's difference between the corresponding components of the vectors. Manhattan distance, also known as Manhattan length, measures the distance between two points measured along axes at right angles. Cosine similarity measures the cosine of the angle between two vectors by first normalizing the vectors to unit length, and then taking the dot product of these normalized vectors. The resulting value ranges from negative one (“−1”) to positive one (“+1”), where a cosine similarity of positive one (“+1”) implies the vectors are perfectly aligned (i.e., an exact match), zero (“0”) denotes orthogonality (i.e., no similarity), and negative one (“−1”) signifies dissimilarity in direction (i.e., complete opposite). Centroid comparator 118 may, in embodiments, be configured to provide, to chemical structure comparator 120, centroid set 138 of centroid feature vectors that satisfy the similarity condition with query feature vector 136.

Chemical structure comparator 120 is configured to determine a compound set 140 of compounds 126 that are associated in index 128 with centroid feature vectors of centroid set 138, and determine whether compound set 140 includes a positive match for query feature vector 134. For instance, chemical structure comparator 120 may, in embodiments, employ index 128 to determine, from among compounds 126, compound set 140 that includes compounds that are associated with centroid feature vectors of centroid set 138. In embodiments, a positive match may include, but is not limited to, a compound in compound set 140 that is an exact match to query feature vector 134, and/or a compound in compound set 140 with a similarity with query feature vector 134 that satisfies a compound similarity condition. Chemical structure comparator 120 may, in embodiments, determine a similarity between a compound of compound set 140 and query feature vector 134 in a similar manner to centroid comparator 118. For instance, chemical structure comparator 120 may determine a difference and/or distance between query feature vector 134 and compound feature vectors representative of compounds of compound set 140, and determine whether the difference and/or distance satisfies a predetermined relationship with a compound similarity threshold. Various functions may be employed by chemical structure comparator 120 to determine a difference and/or distance between query feature vector 132 and centroid feature vectors 124, including, but not limited to, Euclidean distance, Manhattan distance, cosine similarity, and/or the like. In embodiments, chemical structure comparator 120 may directly compare molecular representation 132 with compounds in compound set 140 to determine whether molecular representation 132 is present in compound set 140. For instance, molecular representation 132 may, in embodiments, include a Markush structure, and chemical structure comparator 120 may determine whether any compounds in compound set 140 satisfy the Markush structure represented by molecular representation 132. Chemical structure comparator 120 may be provide results 142 to results processor 122.

Results processor 122 may be configured to receive results 142 and perform one or more actions responsive thereto. For instance, if results 142 include a positive match, results processor 122 may provide the positive match to client 102 for output via UI 108. If results 142 does not include a positive match, results processor 122 may, in embodiments, cause centroid comparator 118 and/or chemical structure comparator 120 to perform a subsequent phase search of chemical structure database 112 based on one or more similarity conditions that may result in a larger search space. In embodiments, results processor 122 may initiate a final phase search of chemical structure database 112 to determine whether previously unsearched portions of chemical structure database 112 include a positive match to query feature vector 134. After one or more search phases without a positive match, results processor 122 may, in embodiments, provide client 102 with an indication that molecular representation 132 is not present in chemical structure database 112.

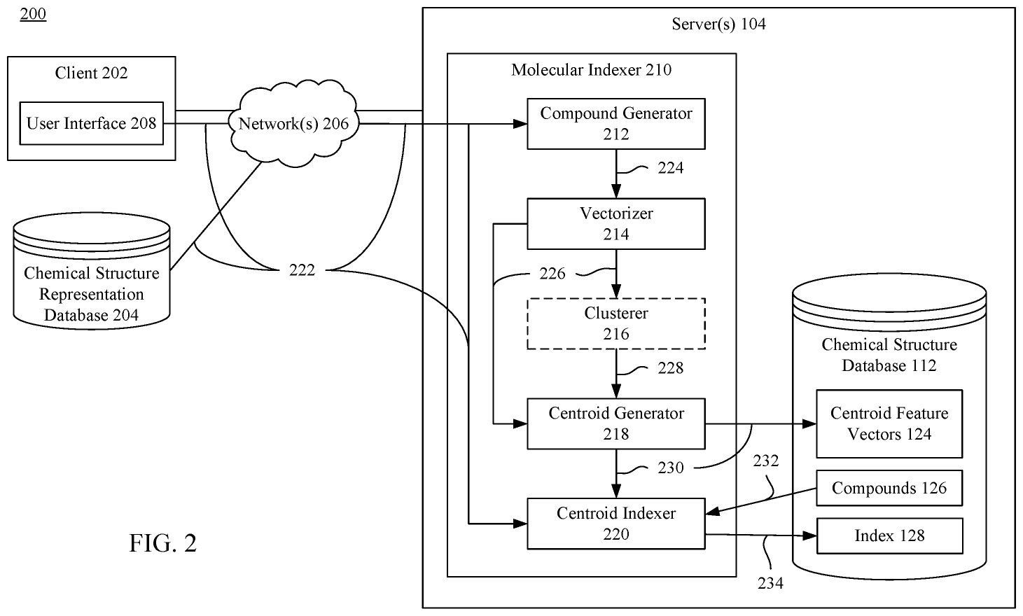

Centroid feature vectors 124 may include centroid feature vectors representative of an approximate centroid or average of a compounds that satisfy a Markush group and/or a cluster thereof. Centroid feature vectors 124 will be described in greater detail in conjunction with FIG. 2 below.

Compounds 126 may include one or more representations of known compounds in chemical structure database 112, including, but not limited to, a machine-readable molecular representation, a Simplified Molecular Input Line Entry System (SMILES) string, a connection table, an atom connectivity matrix, a molfile, a chemical table file, an RGFile, and/or a machine-learning representation. In embodiments, compounds 126 may also include compound feature vectors that are representative of compounds 126. For instance, compound feature vectors may be determined for compounds 126 in a similar manner to the generation of query feature vector 134 by vectorizer 116, as described above.

Index 128 may include one or more associations and/or mappings between centroid feature vectors 124 and compounds 126. For instance, index 128 may associate a compound 126 to one of more centroid feature vectors 124 when compound 126 satisfies a chemical structure representation associated with the one of more centroid feature vectors 124.

Embodiments described herein may operate in various ways index a chemical structure database based on centroids of chemical structure representations, such as, but not limited to, Markush structures. For instance, FIG. 2 shows a block diagram of an example system 200 for indexing a chemical structure database based on centroids of chemical structure representations, in accordance with an embodiment. As shown in FIG. 2, system 200 includes server(s) 104, which includes chemical structure database 112, which includes centroid vectors 124, compounds 126, and index 128. System 200 further includes a client 202 and a chemical structure representation database 204, which are communicatively coupled to server(s) 104 via one or more networks 206. In system 200, client 202 further includes a UI 208, and server(s) 104 further includes a molecular indexer 210, which includes a compound generator 212, a vectorizer 214, a clusterer 216, a centroid generator 218, and a centroid indexer 220.

Network(s) 206 may comprise one or more networks such as local area networks (LANs), wide area networks (WANs), enterprise networks, the Internet, etc., and may include one or more wired and/or wireless portions. In embodiments, network(s) 206 may be distinct from, partially overlap with, and/or be identical to network(s) 106 of FIG. 1. Various example implementations of network(s) 206 are described below in reference to FIG. 8 (e.g., network 804, and/or components thereof).

Client 202 may comprise any type of stationary or mobile processing device, including, but not limited to, a desktop computer, a server, a mobile or handheld device (e.g., a tablet, a personal data assistant (PDA), a smart phone, a laptop, etc.), etc. In embodiments, client 202 may be separate from, overlap with, and/or be identical to client 102 of FIG. 1. In embodiments, client 202 may provide one or more chemical structure representations 222 to molecular indexer 110 to enable molecular indexer 110 to index chemical structure database 112 based on centroids vectors associated with chemical structure representation(s) 222. Various example implementations of client 202 are described below in reference to FIG. 8 (e.g., computing device 802, and/or components thereof).

In embodiments, UI 208 may comprise various interfaces, such as, but not limited to, a user interface (UI), a graphical user interface (GUI), a command-line interface (CLI), an application programming interface (API), and/or the like, to facilitate indexing of chemical structure databases based on centroids associated with chemical structure representations, such as, but not limited to, Markush structures. UI 208 may, in embodiments, enable input of chemical structure representation(s) 222, such as, but not limited to, a machine-readable molecular representation, an RGFile, and/or a machine-learning representation. In embodiments, UI 202 may enable chemical structure representation(s) 222 to be input through various ways, such as, but not limited to, by uploading a file containing the molecular representation, by inputting the molecular representation as text, by drawing a graphical molecular representation, by verbally dictating or describing the molecular representation, and/or the like. In embodiments, UI 208 may be separate from, overlap with, and/or be identical to UI 108 of FIG. 1. Various example implementations of UI 208 are described below in reference to FIG. 8 (e.g., application 814, input device(s) 830, output device(s) 850, and/or components thereof).

Chemical structure representation database 204 may include one or more databases storing known Markush structures, such as, but not limited to, a database of patented Markush structures, a database of Markush structures described in publications, a database of RGFiles representative of Markush structures, and/or the like. In embodiments, chemical structure representation database 204 may provide chemical structure representation(s) 222 to molecular indexer 210 to enable molecular indexer 210 to index chemical structure database 112 based on centroids vectors associated with chemical structure representation(s) 222.

Molecular indexer 210 is configured to index chemical structure database 112 based on centroids vectors associated with chemical structure representation(s) 222. In embodiments, molecular indexer 210 receives a chemical structure representation(s) 222 from one or more of client 202 and/or chemical structure representation database 204, determine a centroid feature vector 232 based on one or more randomly generated compounds 224 that satisfy the Markush structure associated with chemical structure representation(s) 222, and index chemical structure database 112 by associating compounds 126 that satisfy the Markush structure with centroid feature vector 232 associated with the Markush structure.

Compound generator 212 is configured to receive chemical structure representation(s) 222 from one or more of client 202 and/or chemical structure representation database 204 and generate a plurality of random compounds that satisfy the Markush structure associated with chemical structure representation(s) 222. For instance, compound generator 212 may, in embodiments, generate random compound(s) 224 by randomly selecting a permissible substituent for each R-group in the Markush structure associated with chemical structure representation(s) 222. In embodiments, random compound(s) 224 may include representations in various formats, such as, but not limited to, a machine-readable molecular representation, a Simplified Molecular Input Line Entry System (SMILES) string, a connection table, an atom connectivity matrix, a molfile, a chemical table file, and/or a machine-learning representation. Compound generator 212 may provide random compound(s) 224 to vectorizer 214.

Vectorizer 214 is configured to receive random compound(s) 224 from compound generator 212 and generate one or more compound feature vectors 226 that are representative of random compound(s) 224. In embodiments, vectorizer 214 may operate in a similar and/or the same manner as vectorizer 116 of FIG. 1. In embodiments, compound feature vector(s) 226 are numerical vectors that capture various molecular properties of random compound(s) 224. For instance, vectorizer 214 may, in embodiments, encode structural information, such as, but not limited to, atom types, bond types, and/or their spatial arrangements, into a numerical vector. In embodiments, vectorizer 214 may encode additional molecular properties into query feature vector 134, including, but not limited to, patterns of atoms and/or bonds, molecular weight, solubility, electronegativity, molecular conformations, spatial arrangements, and/or the like. In embodiments, vectorizer 214 may employ one or more hash functions to convert random compound(s) 224 into compound feature vector(s) 226. In embodiments, vectorizer 214 may employ an ML encoder that is trained on a large set of chemical structures. For instance, vectorizer 214 may employ an ML encoder to transform random compound(s) 224 into a condensed vector representation, such as compound feature vector(s) 226. In embodiments, an ML encoder may be trained by iteratively optimizing parameters to learn meaningful representations of input molecular representations by minimizing, over multiple iterations, a loss function that quantifies the difference between a generated representation and a target representation. In embodiments, the loss function may be based on whether or not molecular representations satisfy a common Markush structure. Vectorizer 214 may be configured to provide compound feature vector(s) 226 to clusterer 216 and/or centroid generator 218.

Clusterer 216 is configured to receive compound feature vector(s) 226 from vectorizer 214 and group compound feature vector(s) 226 into one or more compound clusters 228. For instance, clusterer 216 may, in embodiments, employ an unsupervised machine learning algorithm to organize similar compound feature vector(s) 226 into cluster(s) 228 based on the characteristics of random compound(s) 224 that are encoded in compound feature vector(s) 226. In embodiments, clusterer 216 may employ various algorithms, such as, but not limited to, K-means, hierarchical clustering, and/or DBSCAN, to partition compound feature vector(s) 226 into cluster(s) 228 by minimizing intra-cluster differences and/or maximizing inter-cluster dissimilarities. In embodiments, clusterer 216 may be omitted from system 200, and/or incorporated into centroid generator 218. In embodiments, clusterer 216 may provide a single cluster 228 based on determining that the intra-cluster differences between compound feature vector(s) 226 satisfy one or more conditions, such as, but not limited to, an intra-cluster difference satisfying a predetermined relationship with an intra-cluster difference threshold, and/or a cluster size satisfying a predetermined relationship with a cluster size threshold. Clusterer 216 may, in embodiments, provide cluster(s) 228 to centroid generator 218.

Centroid generator 218 is configured to calculate one or more centroid feature vectors 230 based on compound feature vector(s) 226 received from vectorizer 214 and/or cluster(s) 228 received from clusterer 216. For instance, centroid generator 218 may, in embodiments, determine centroid feature vector(s) 230 for cluster(s) 228 by computing, for each particular cluster 228, the mean or average of each dimension of compound feature vector(s) 226 belonging to the particular cluster 228. In embodiments, a centroid feature vector C of a cluster comprising n compound feature vectors v₁, v₂, . . . , vₙ, may be represented mathematically as:

$${C}_{i} = \frac{1}{n} {\sum }_{j = 1}^{n} {v}_{ij} , \qquad ( Eq . 1 )$$

where vⱼᵢ represents the value of dimension i in vector vⱼ, and Cᵢ represents the i-th dimension of the centroid feature vector C.

In embodiments, centroid generator 218 may iteratively calculate centroid feature vector(s) 230 by calculating, in a first iteration, an initial centroid feature vector based on an initial set of compound feature vector(s) 226, and recalculating the centroid feature vector based on additional compound feature vector(s) 226 associated with additional random compound(s) 224 that satisfy the chemical structure representation(s) 222 until a convergence condition is met. For instance, centroid generator 218 may, in embodiments, determine during a second iteration that the convergence condition is not met, and initiate a third iteration. In a third iteration, centroid generator 218 may, in embodiments, recalculate the centroid feature vector based on the previous centroid feature vector and one or more of: additional random compound(s) 224, compound feature vector(s) 226, and/or cluster(s) 228 received from compound generator 212, vectorizer 214, and/or clusterer 216, respectively, and determine whether the recalculated centroid feature vector satisfies the convergence condition. In embodiments, the convergence condition may include, but is not limited to, the difference in centroid feature vectors between temporally adjacent iterations satisfies a percentage difference threshold, the difference in centroid feature vectors between temporally adjacent iterations satisfies an absolute difference threshold, the difference in centroid feature vectors between temporally adjacent iterations satisfies a distance threshold, the number of iterations exceeds an iteration threshold, and/or the like. In embodiments, centroid generator 218 may provide centroid feature vector(s) 230 to chemical structure database 112 and/or centroid indexer 220 upon satisfaction of the convergence condition. For instance, centroid generator 218 may store centroid feature vector(s) 230 in chemical structure database 112 as centroid feature vectors 124.

Centroid indexer 220 is configured to index chemical structure database 112 by associating centroid feature vector(s) 230 with compounds 126 that satisfy chemical structure representation(s) 222, and/or a cluster thereof. For instance, centroid indexer 220 may, in embodiments, receive a centroid feature vector 230 from centroid generator 218, determine, from among compounds 126, one or more compounds 232 that satisfy chemical structure representation(s) 222, and generate one or more associations 234 that associate compound(s) 232 with centroid feature vector 230 in index 128. In embodiments, centroid indexer 220 may associate compound(s) 232 with a centroid feature vector 230 of a particular cluster 228 by determining a smallest distance or difference between compound(s) 232 and centroid feature vector(s) 230. In embodiments, the distance or difference between compound(s) 232 and centroid feature vector(s) 230 may be determined through various methods, including, but not limited to, calculating a Euclidean distance, calculating a Manhattan distance, calculating a cosine similarity, and/or the like. Centroid indexer 220 may, in embodiments, store association(s) 234 in index 128.

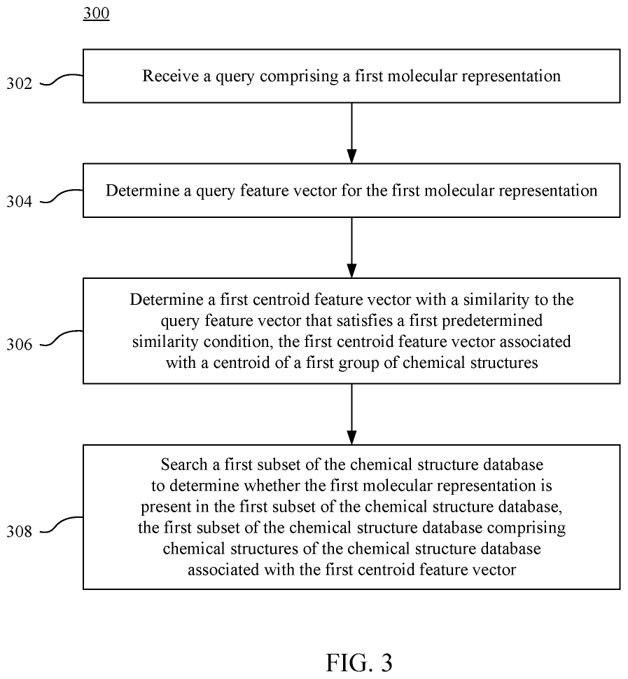

Embodiments described herein may operate in various ways to search a subset of a chemical structure database based on centroids. For instance, FIG. 3 depicts a flowchart 300 of a process for searching a first subset of a chemical structure database based on centroids, in accordance with an embodiment. Server(s) 104, molecular searcher 110, request processor 114, vectorizer 116, centroid comparator 118, chemical structure comparator 120, and/or results processor 122 of FIG. 1 may operate according to flowchart 300, for example. Note that not all steps of flowchart 300 may need to be performed in all embodiments, and in some embodiments, the steps of flowchart 300 may be performed in different orders than shown. Flowchart 300 is described as follows with respect to FIG. 1 for illustrative purposes.

Flowchart 300 starts at step 302. In step 302, a query comprising a first molecular representation is received. For example, request processor 114 may receive a query 130 from client 102 over network(s) 106. In embodiments, request processor 114 may extract molecular representation 132 from query 130 and provide molecular representation 132 to vectorizer 116. In embodiments, molecular representation 132 may include, but is not limited to, a machine-readable molecular representation, a Simplified Molecular Input Line Entry System (SMILES) string, a connection table, an atom connectivity matrix, a molfile, a chemical table file, an RGFile, and/or a machine-learning representation.

In step 304, a query feature vector is determined for the first molecular representation. For example, vectorizer 116 may determine a query feature vector 134 based on molecular representation 132. In embodiments, vectorizer 116 may generate a query feature vector 134 that is representative of molecular representation 132. In embodiments, query feature vector 134 is a numerical vector that captures various molecular properties of molecular representation 132. For instance, vectorizer 116 may, in embodiments, encode structural information and/or molecular properties of molecular representation 132 into query feature vector 134. In embodiments, vectorizer 116 may employ an ML encoder and/or one or more hash functions to encode molecular representation 132 into query feature vector 134. Vectorizer 116 may, in embodiments, provide query feature vector 134 to centroid comparator 118 and/or chemical structure comparator 120.

In step 306, a first centroid feature vector with a similarity to the query feature vector that satisfies a first predetermined similarity condition is determined, the first centroid feature vector associated with a centroid of a first group of chemical structures associated with a first chemical structure representation. For example, centroid comparator 118 may determine, from among centroid feature vector(s) 136, a first centroid set 138 of centroid feature vectors that satisfy a first predetermined similarity condition, such as, but not limited to, a similarity threshold, a distance threshold, a difference threshold, and/or the like, to query feature vector 134. In embodiments, centroid feature vector(s) 136 may include some or all of centroid feature vectors 124. For instance, in embodiments, centroid comparator 118 may receive, as centroid feature vector(s) 136, centroid feature vectors 124 associated with chemical structure representations that satisfy one or more filtering conditions, such as, but not limited to, chemical structure representations that include a particular chemical element and/or element group, chemical structure representations that do not include a particular chemical element and/or element group, and/or the like. Centroid comparator 118 may, in embodiments, provide first centroid set 138 to chemical structure comparator 120.

In step 308, a first subset of the chemical structure database is searched to determine whether the first molecular representation is present in the first subset of the chemical structure database, the first subset of the chemical structure database comprising chemical structures of the chemical structure database associated with the first centroid feature vector. For example, chemical structure comparator 120 may employ index 128 to determine a first compound set 140 that includes compounds 126 that are associated with centroid feature vectors of first centroid set 138, and search for molecular representation 132 in first compound set 140. In embodiments, a positive match may include, but is not limited to, a compound in first compound set 140 that is an exact match to query feature vector 134, and/or a compound in first compound set 140 with a similarity with query feature vector 134 that satisfies a compound similarity condition. Chemical structure comparator 120 may, in embodiments, determine a difference and/or distance between query feature vector 134 and compound feature vectors representative of compounds of first compound set 140, and determine whether the difference and/or distance satisfies a predetermined relationship with a compound similarity threshold. In embodiments, chemical structure comparator 120 may directly compare molecular representation 132 with compounds in first compound set 140 to determine whether molecular representation 132 is present in compound set 140. For instance, molecular representation 132 may, in embodiments, include a Markush structure, and chemical structure comparator 120 may determine whether any compounds in first compound set 140 satisfy the Markush structure represented by molecular representation 132. Chemical structure comparator 120 may be provide results 142 to results processor 122.

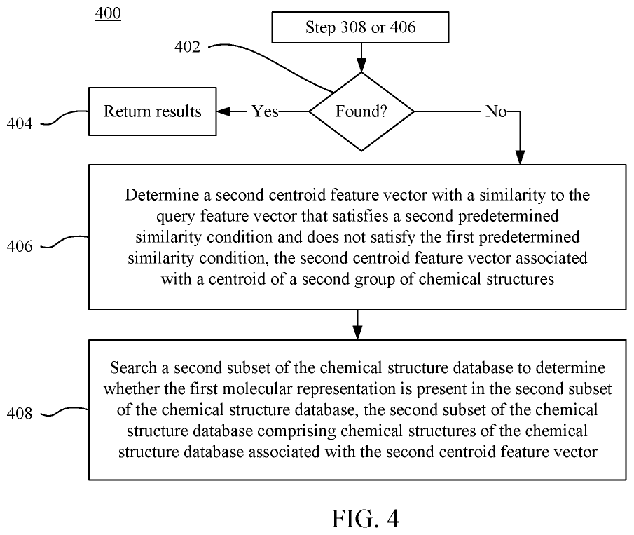

Embodiments described herein may operate in various ways to search a second subset of a chemical structure database based on centroids. For instance, FIG. 4 depicts a flowchart 400 of a process for searching a second subset of a chemical structure database based on centroids, in accordance with an embodiment. Server(s) 104, molecular searcher 110, request processor 114, vectorizer 116, centroid comparator 118, chemical structure comparator 120, and/or results processor 122 of FIG. 1 may operate according to flowchart 400, for example. Note that not all steps of flowchart 400 may need to be performed in all embodiments, and in some embodiments, the steps of flowchart 400 may be performed in different orders than shown. Flowchart 400 is described as follows with respect to FIG. 1 for illustrative purposes.

Flowchart 400 starts at step 402. In embodiments, the process of flowchart 400 executes after the execution of step 308 of FIG. 3, and/or step 408 of FIG. 4. In step 402, it is determined whether step 308 of FIG. 3 or 408 of FIG. 4 produces a positive match. For example, results processor 122 may receive results 142 from chemical structure comparator 120 and determine whether results 142 includes a positive match. If results 142 includes a positive match, flowchart 400 proceeds to step 404, otherwise, flowchart 400 proceeds to step 406.

In step 404, results are returned. For example, results processor 122 may provide results 142 to client 102 for output via UI 108.

In step 406, a second centroid feature vector with a similarity to the query feature vector that satisfies a second predetermined similarity condition and does not satisfy the first predetermined similarity condition is determined, the second centroid feature vector associated with a centroid of a second group of chemical structures associated with a second chemical structure representation. For example, results processor 122 may cause centroid comparator 118 to determine, from among centroid feature vector(s) 136, a second centroid set 138 of centroid feature vectors that satisfy a second predetermined similarity condition, but not the first predetermined similarity condition, to query feature vector 134. In embodiments, the second predetermined similarity condition includes, but is not limited to, a similarity threshold, a distance threshold, a difference threshold, and/or the like, that results in a larger search space than the first predetermined similarity condition. For instance, the second predetermined similarity condition may, in embodiment, result in second centroid set 138 including centroid feature vectors that were not present in first centroid set 138. Centroid comparator 118 may, in embodiments, provide second centroid set 138 to chemical structure comparator 120.

In step 408, a second subset of the chemical structure database is searched to determine whether the first molecular representation is present in the second subset of the chemical structure database, the second subset of the chemical structure database comprising chemical structures of the chemical structure database associated with the second centroid feature vector. For example, chemical structure comparator 120 may employ index 128 to determine second compound set 140 that includes compounds 126 that are associated with centroid feature vectors of second centroid set 138, and search for molecular representation 132 in second compound set 140. In embodiments, a positive match may include, but is not limited to, a compound in second compound set 140 that is an exact match to query feature vector 134, and/or a compound in second compound set 140 with a similarity with query feature vector 134 that satisfies a compound similarity condition. Chemical structure comparator 120 may, in embodiments, determine a difference and/or distance between query feature vector 134 and compound feature vectors representative of compounds of second compound set 140, and determine whether the difference and/or distance satisfies a predetermined relationship with a compound similarity threshold. In embodiments, chemical structure comparator 120 may directly compare molecular representation 132 with compounds in second compound set 140 to determine whether molecular representation 132 is present in second compound set 140. For instance, molecular representation 132 may, in embodiments, include a Markush structure, and chemical structure comparator 120 may determine whether any compounds in second compound set 140 satisfy the Markush structure represented by molecular representation 132. Chemical structure comparator 120 may be provide results 142 to results processor 122.

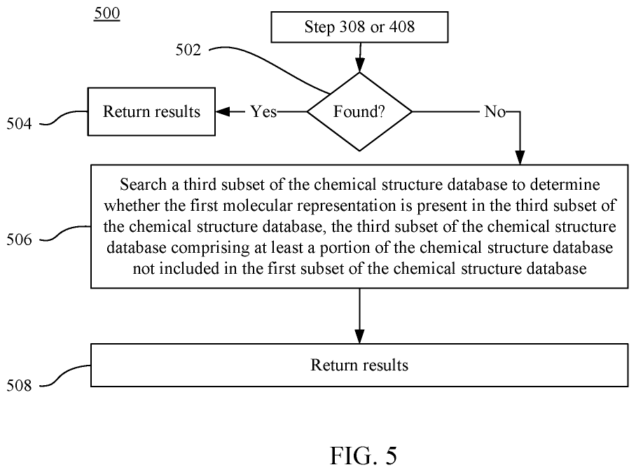

Embodiments described herein may operate in various ways to search a third subset of a chemical structure database based on centroids. For instance, FIG. 5 depicts a flowchart 500 of a process for searching a third subset of a chemical structure database based on centroids, in accordance with an embodiment. Server(s) 104, molecular searcher 110, request processor 114, vectorizer 116, centroid comparator 118, chemical structure comparator 120, and/or results processor 122 of FIG. 1 may operate according to flowchart 500, for example. Note that not all steps of flowchart 500 may need to be performed in all embodiments, and in some embodiments, the steps of flowchart 500 may be performed in different orders than shown. Flowchart 500 is described as follows with respect to FIG. 1 for illustrative purposes.

Flowchart 500 starts at step 502. In embodiments, the process of flowchart 500 executes after the execution of step 308 of FIG. 3, and/or step 408 of FIG. 4. In step 502, it is determined whether step 308 of FIG. 3 or 408 of FIG. 4 produces a positive match. For example, results processor 122 may receive results 142 from chemical structure comparator 120 and determine whether results 142 includes a positive match. If results 142 includes a positive match, flowchart 500 proceeds to step 504, else flowchart 500 proceeds to step 506.

In step 504, results are returned. For example, results processor 122 may provide results 142 to client 102 for output via UI 108.

In step 506, a third subset of the chemical structure database is searched to determine whether the first molecular representation is present in the third subset of the chemical structure database, the third subset of the chemical structure database comprising at least a portion of the chemical structure database not included in the first subset of the chemical structure database. For example, results processor 122 may cause chemical structure comparator 120 to determine a final compound set 140 that includes compounds 126 that were not included in previous search phases, and determine whether a positive match for molecular representation 132 exists in the compounds that were not included in previous search phases. In embodiments, chemical structure comparator 120 may provide results 142 to results processor 122.

In step 508, results are returned. For example, results processor 122 may provide results 142 to client 102 for output via UI 108. If results 142 does not include a positive result, results processor 122 may provide a negative result to client 102 for output via UI 108 to indicate that molecular representation 132 is not present in chemical structure database 112.

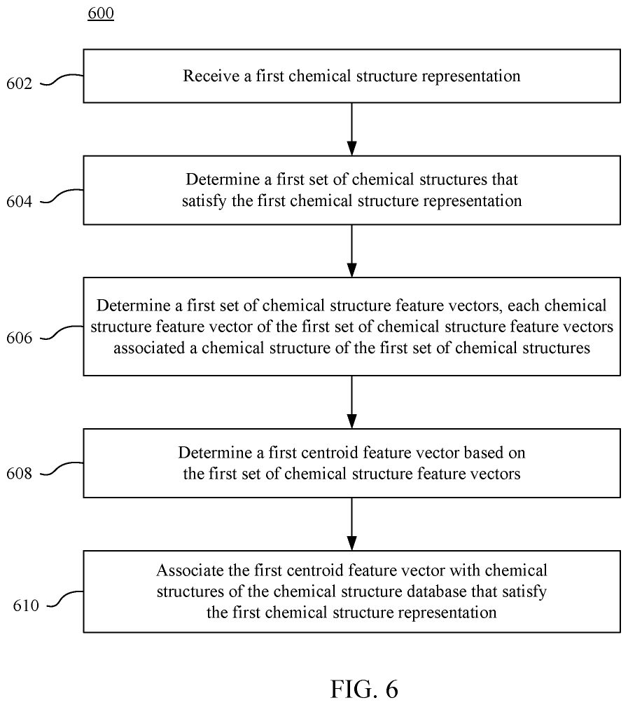

Embodiments described herein may operate in various ways to index a chemical structure database based on a centroid of a chemical structure representation, such as, but not limited to, a Markush structure. For instance, FIG. 6 depicts a flowchart 600 of a process for indexing a chemical structure database based on a centroid of a chemical structure representation, in accordance with an embodiment. Server(s) 104, molecular indexer 210, compound generator 212, vectorizer 214, clusterer 216, centroid generator 218, and/or centroid indexer 220 of FIG. 2 may operate according to flowchart 600, for example. Note that not all steps of flowchart 600 may need to be performed in all embodiments, and in some embodiments, the steps of flowchart 600 may be performed in different orders than shown. Flowchart 600 is described as follows with respect to FIG. 2 for illustrative purposes.

Flowchart 600 starts at step 602. In step 602, a first chemical structure representation is received. For example, compound generator 212 may receive, from client 202 and/or chemical structure representation database 204, chemical structure representation(s) 222.

In step 604, a first set of chemical structures that satisfy the first chemical structure representation is determined. For example, compound generator 212 may generate random compound(s) 224 by randomly selecting a permissible substituent for each R-group in the Markush structure associated with chemical structure representation(s) 222. In embodiments, random compound(s) 224 may include representations in various formats, such as, but not limited to, a machine-readable molecular representation, a Simplified Molecular Input Line Entry System (SMILES) string, a connection table, an atom connectivity matrix, a molfile, a chemical table file, and/or a machine-learning representation. Compound generator 212 may, in embodiments, provide random compound(s) 224 to vectorizer 214.

In step 606, a first set of chemical structure feature vectors is determined, each chemical structure feature vector of the first set of chemical structure feature vectors associated a chemical structure of the first set of chemical structures. For example, vectorizer 214 may generate compound feature vector(s) 226 that are representative of random compound(s) 224. In embodiments, compound feature vector(s) 226 are numerical vectors that capture various molecular properties of random compound(s) 224. In embodiments, vectorizer 214 may employ an ML encoder and/or one or more hash functions to convert random compound(s) 224 into compound feature vector(s) 226. Vectorizer 214 may, in embodiments, provide compound feature vector(s) 226 to clusterer 216 and/or centroid generator 218.

In step 608, a first centroid feature vector is determined based on the first set of chemical structure feature vectors. For example, centroid generator 218 may determine a centroid feature vector 230 based on compound feature vector(s) 226 by computing the mean or average of each dimension of compound feature vector(s) 226. In embodiments, centroid generator 218 may provide centroid feature vector 230 to chemical structure database 112 and/or centroid indexer 220. For instance, centroid generator 218 may store centroid feature vector(s) 230 in chemical structure database 112 as centroid feature vectors 124.

In step 610, the first centroid feature vector is associated with chemical structures of the chemical structure database that satisfies the first chemical structure representation. For example, centroid indexer 220 may associate centroid feature vector 230 with compounds 126 that satisfy chemical structure representation(s) 222. For instance, centroid indexer 220 may, in embodiments, receive a centroid feature vector 230 from centroid generator 218, determine, from among compounds 126, compound(s) 232 that satisfy chemical structure representation(s) 222, and generate association(s) 234 that associate compound(s) 232 with centroid feature vector 230 in index 128. In embodiments, centroid indexer 220 may associate compound(s) 232 with a centroid feature vector 230 of a particular cluster 228 by determining a smallest distance or difference between compound(s) 232 and centroid feature vector(s) 230. Centroid indexer 220 may, in embodiments, store association(s) 234 in index 128.

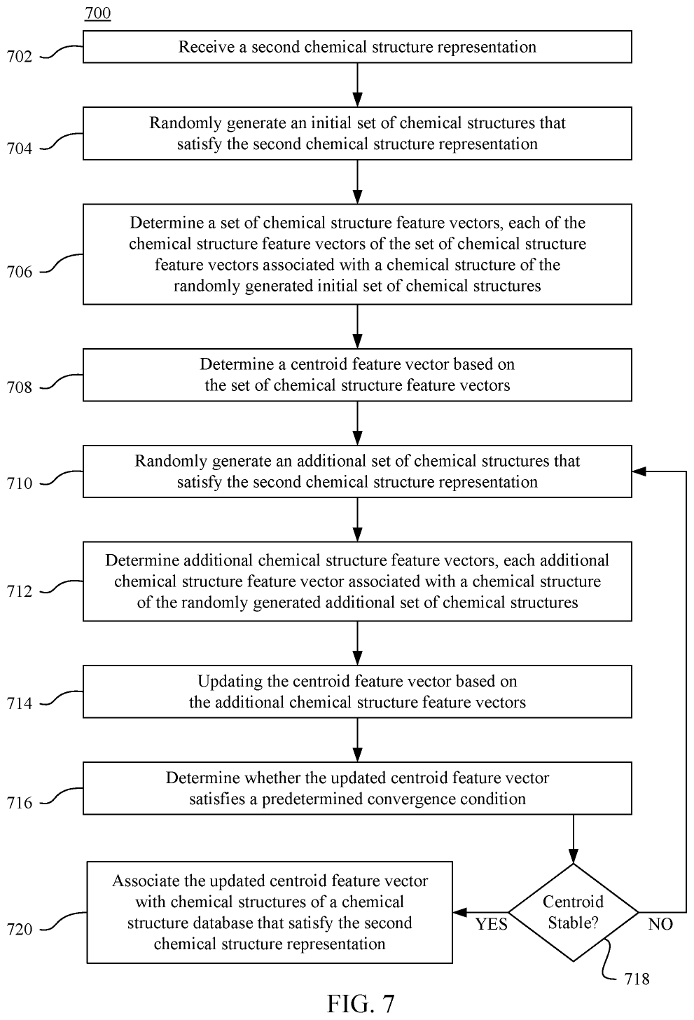

Embodiments described herein may operate in various ways to index a chemical structure database based on a centroid of a chemical structure representation, such as, but not limited to, a Markush structure. For instance, FIG. 7 depicts a flowchart 700 of a process for indexing a chemical structure database based on a centroid of a chemical structure representation, in accordance with an embodiment. Server(s) 104, molecular indexer 210, compound generator 212, vectorizer 214, clusterer 216, centroid generator 218, and/or centroid indexer 220 of FIG. 2 may operate according to flowchart 700, for example. Note that not all steps of flowchart 700 may need to be performed in all embodiments, and in some embodiments, the steps of flowchart 700 may be performed in different orders than shown. Flowchart 700 is described as follows with respect to FIG. 2 for illustrative purposes.

Flowchart 700 starts at step 702. In step 702, a second chemical structure representation is received. For example, compound generator 212 may receive, from client 202 and/or chemical structure representation database 204, chemical structure representation(s) 222.

In step 704, an initial set of chemical structures that satisfy the second chemical structure representation is generated. For example, compound generator 212 may generate random compound(s) 224 by randomly selecting a permissible substituent for each R-group in the Markush structure associated with chemical structure representation(s) 222. In embodiments, random compound(s) 224 may include representations in various formats, such as, but not limited to, a machine-readable molecular representation, a Simplified Molecular Input Line Entry System (SMILES) string, a connection table, an atom connectivity matrix, a molfile, a chemical table file, and/or a machine-learning representation. Compound generator 212 may, in embodiments, provide random compound(s) 224 to vectorizer 214.

In step 706, a second set of chemical structure feature vectors are determined, each of the chemical structure feature vectors of the second set of chemical structure feature vectors associated with a chemical structure of the randomly generated initial set of chemical structures. For example, vectorizer 214 may generate compound feature vector(s) 226 that are representative of random compound(s) 224. In embodiments, compound feature vector(s) 226 are numerical vectors that capture various molecular properties of random compound(s) 224. In embodiments, vectorizer 214 may employ an ML encoder and/or one or more hash functions to convert random compound(s) 224 into compound feature vector(s) 226. Vectorizer 214 may, in embodiments, provide compound feature vector(s) 226 to clusterer 216 and/or centroid generator 218.

In step 708, a centroid feature vector is determined based on the set of chemical structure feature vectors. For example, centroid generator 218 may determine a centroid feature vector 230 based on compound feature vector(s) 226 by computing the mean or average of each dimension of compound feature vector(s) 226. In embodiments, centroid generator 218 may provide centroid feature vector 230 to chemical structure database 112 and/or centroid indexer 220. For instance, centroid generator 218 may store centroid feature vector(s) 230 in chemical structure database 112 as centroid feature vectors 124.

In step 710, an additional set of chemical structures that satisfy the second chemical structure representation is randomly generated. For example, compound generator 212 may generate additional random compound(s) 224 by randomly selecting a permissible substituent for each R-group in the Markush structure associated with chemical structure representation(s) 222. Compound generator 212 may, in embodiments, provide additional random compound(s) 224 to vectorizer 214.

In step 712, additional chemical structure feature vectors are determined, each additional chemical structure feature vector associated with a chemical structure of the randomly generated additional set of chemical structures. For example, vectorizer 214 may generate additional compound feature vector(s) 226 that are representative of additional random compound(s) 224. Vectorizer 214 may, in embodiments, provide additional compound feature vector(s) 226 to clusterer 216 and/or centroid generator 218.

In step 714, the centroid feature vector is updated based on the additional chemical structure feature vectors. For example, centroid generator 218 may determine an updated centroid feature vector 230 based on the previous centroid feature vector and additional compound feature vector(s) 226 by computing the mean or average of each dimension of the previous centroid feature vector and additional compound feature vector(s) 226.

In step 716, it is determined whether the updated centroid feature vector satisfies a predetermined convergence condition. For example, centroid generator 218 may determine whether updated centroid feature vector 230 satisfies a convergence condition, such as, but is not limited to, the difference in centroid feature vectors between temporally adjacent iterations satisfies a percentage difference threshold, the difference in centroid feature vectors between temporally adjacent iterations satisfies an absolute difference threshold, the difference in centroid feature vectors between temporally adjacent iterations satisfies a distance threshold, the number of iterations exceeds an iteration threshold, and/or the like. In embodiments, centroid generator 218 may provide updated centroid feature vector 230 to chemical structure database 112 and/or centroid indexer 220 upon satisfaction of the convergence condition.

In step 718, if the updated centroid feature vector satisfies the predetermined convergence condition, flowchart 700 proceeds to step 720, otherwise, flowchart 700 returns to step 710 where steps 710-718 are executed again to further update centroid feature vector 230.

In step 720, the updated centroid feature vector is associated with chemical structures of a chemical structure database that satisfy the second chemical structure representation. For example, centroid indexer 220 may receive updated centroid feature vector 230 from centroid generator 218, determine, from among compounds 126, compound(s) 232 that satisfy chemical structure representation 222, and generate association(s) 234 that associate compound(s) 232 with updated centroid feature vector 230 in index 128. In embodiments, centroid indexer 220 may associate compound(s) 232 with a centroid feature vector 230 of a particular cluster 228 by determining a smallest distance or difference between compound(s) 232 and centroid feature vector(s) 230. Centroid indexer 220 may, in embodiments, store association(s) 234 in index 128.

### III. Example Mobile Device and Computer System Implementation

The systems and methods described above in reference to FIGS. 1-7, including client 102, server(s) 104, network(s) 106, UI 108, molecular searcher 110, chemical structure database 112, request processor 114, vectorizer 116, centroid comparator 118, chemical structure comparator 120, results processor 122, centroid feature vectors 124, compounds 126, index 128, client 202, chemical structure representation database 204, network(s) 206, UI 208, molecular indexer 210, compound generator 212, vectorizer 214, clusterer 216, centroid generator 218, centroid indexer 220, and/or each of the components described therein, and/or the steps of flowcharts 300, 400, 500, 600, and/or 700 may be implemented in hardware, or hardware combined with one or both of software and/or firmware. For example, client 102, server(s) 104, network(s) 106, UI 108, molecular searcher 110, chemical structure database 112, request processor 114, vectorizer 116, centroid comparator 118, chemical structure comparator 120, results processor 122, centroid feature vectors 124, compounds 126, index 128, client 202, chemical structure representation database 204, network(s) 206, UI 208, molecular indexer 210, compound generator 212, vectorizer 214, clusterer 216, centroid generator 218, centroid indexer 220, and/or each of the components described therein, and/or the steps of flowcharts 300, 400, 500, 600, and/or 700 may be each implemented as computer program code/instructions configured to be executed in one or more processors and stored in a computer readable storage medium. 1. Alternatively, client 102, server(s) 104, network(s) 106, UI 108, molecular searcher 110, chemical structure database 112, request processor 114, vectorizer 116, centroid comparator 118, chemical structure comparator 120, results processor 122, centroid feature vectors 124, compounds 126, index 128, client 202, chemical structure representation database 204, network(s) 206, UI 208, molecular indexer 210, compound generator 212, vectorizer 214, clusterer 216, centroid generator 218, centroid indexer 220, and/or each of the components described therein, and/or the steps of flowcharts 300, 400, 500, 600, and/or 700 may be implemented in one or more SoCs (system on chip). An SoC may include an integrated circuit chip that includes one or more of a processor (e.g., a central processing unit (CPU), microcontroller, microprocessor, digital signal processor (DSP), etc.), memory, one or more communication interfaces, and/or further circuits, and may optionally execute received program code and/or include embedded firmware to perform functions.

Embodiments disclosed herein may be implemented in one or more computing devices that may be mobile (a mobile device) and/or stationary (a stationary device) and may include any combination of the features of such mobile and stationary computing devices. Examples of computing devices in which embodiments may be implemented are described as follows with respect to FIG. 8. FIG. 8 shows a block diagram of an exemplary computing environment 800 that includes a computing device 802. In some embodiments, computing device 802 is communicatively coupled with devices (not shown in FIG. 8) external to computing environment 800 via network 804. Network 804 comprises one or more networks such as local area networks (LANs), wide area networks (WANs), enterprise networks, the Internet, etc., and may include one or more wired and/or wireless portions. Network 804 may additionally or alternatively include a cellular network for cellular communications. Computing device 802 is described in detail as follows

Computing device 802 can be any of a variety of types of computing devices. For example, computing device 802 may be a mobile computing device such as a handheld computer (e.g., a personal digital assistant (PDA)), a laptop computer, a tablet computer (such as an Apple iPad™), a hybrid device, a notebook computer (e.g., a Google Chromebook™ by Google LLC), a netbook, a mobile phone (e.g., a cell phone, a smart phone such as an Apple® iPhone® by Apple Inc., a phone implementing the Google® Android™ operating system, etc.), a wearable computing device (e.g., a head-mounted augmented reality and/or virtual reality device including smart glasses such as Google® Glass™, Oculus Rift® of Facebook Technologies, LLC, etc.), or other type of mobile computing device. Computing device 802 may alternatively be a stationary computing device such as a desktop computer, a personal computer (PC), a stationary server device, a minicomputer, a mainframe, a supercomputer, etc.

As shown in FIG. 8, computing device 802 includes a variety of hardware and software components, including a processor 810, a storage 820, one or more input devices 830, one or more output devices 850, one or more wireless modems 860, one or more wired interfaces 880, a power supply 882, a location information (LI) receiver 884, and an accelerometer 886. Storage 820 includes memory 856, which includes non-removable memory 822 and removable memory 824, and a storage device 890. Storage 820 also stores an operating system 812, application programs 814, and application data 816. Wireless modem(s) 860 include a Wi-Fi modem 862, a Bluetooth modem 864, and a cellular modem 866. Output device(s) 850 includes a speaker 852 and a display 854. Input device(s) 830 includes a touch screen 832, a microphone 834, a camera 836, a physical keyboard 838, and a trackball 840. Not all components of computing device 802 shown in FIG. 8 are present in all embodiments, additional components not shown may be present, and any combination of the components may be present in a particular embodiment. These components of computing device 802 are described as follows.

A single processor 810 (e.g., central processing unit (CPU), microcontroller, a microprocessor, signal processor, ASIC (application specific integrated circuit), and/or other physical hardware processor circuit) or multiple processors 810 may be present in computing device 802 for performing such tasks as program execution, signal coding, data processing, input/output processing, power control, and/or other functions. Processor 810 may be a single-core or multi-core processor, and each processor core may be single-threaded or multithreaded (to provide multiple threads of execution concurrently). Processor 810 is configured to execute program code stored in a computer readable medium, such as program code of operating system 812 and application programs 814 stored in storage 820. Operating system 812 controls the allocation and usage of the components of computing device 802 and provides support for one or more application programs 814 (also referred to as “applications” or “apps”). Application programs 814 may include common computing applications (e.g., e-mail applications, calendars, contact managers, web browsers, messaging applications), further computing applications (e.g., word processing applications, mapping applications, media player applications, productivity suite applications), one or more machine learning (ML) models, as well as applications related to the embodiments disclosed elsewhere herein.

Any component in computing device 802 can communicate with any other component according to function, although not all connections are shown for ease of illustration. For instance, as shown in FIG. 8, bus 806 is a multiple signal line communication medium (e.g., conductive traces in silicon, metal traces along a motherboard, wires, etc.) that may be present to communicatively couple processor 810 to various other components of computing device 802, although in other embodiments, an alternative bus, further buses, and/or one or more individual signal lines may be present to communicatively couple components. Bus 806 represents one or more of any of several types of bus structures, including a memory bus or memory controller, a peripheral bus, an accelerated graphics port, and a processor or local bus using any of a variety of bus architectures.

Storage 820 is physical storage that includes one or both of memory 856 and storage device 890, which store operating system 812, application programs 814, and application data 816 according to any distribution. Non-removable memory 822 includes one or more of RAM (random access memory), ROM (read only memory), flash memory, a solid-state drive (SSD), a hard disk drive (e.g., a disk drive for reading from and writing to a hard disk), and/or other physical memory device type. Non-removable memory 822 may include main memory and may be separate from or fabricated in a same integrated circuit as processor 810. As shown in FIG. 8, non-removable memory 822 stores firmware 818, which may be present to provide low-level control of hardware. Examples of firmware 818 include BIOS (Basic Input/Output System, such as on personal computers) and boot firmware (e.g., on smart phones). Removable memory 824 may be inserted into a receptacle of or otherwise coupled to computing device 802 and can be removed by a user from computing device 802. Removable memory 824 can include any suitable removable memory device type, including an SD (Secure Digital) card, a Subscriber Identity Module (SIM) card, which is well known in GSM (Global System for Mobile Communications) communication systems, and/or other removable physical memory device type. One or more of storage device 890 may be present that are internal and/or external to a housing of computing device 802 and may or may not be removable. Examples of storage device 890 include a hard disk drive, a SSD, a thumb drive (e.g., a USB (Universal Serial Bus) flash drive), or other physical storage device.

One or more programs may be stored in storage 820. Such programs include operating system 812, one or more application programs 814, and other program modules and program data. Examples of such application programs may include, for example, computer program logic (e.g., computer program code/instructions) for implementing one or more of client 102, server(s) 104, network(s) 106, UI 108, molecular searcher 110, chemical structure database 112, request processor 114, vectorizer 116, centroid comparator 118, chemical structure comparator 120, results processor 122, centroid feature vectors 124, compounds 126, index 128, client 202, chemical structure representation database 204, network(s) 206, UI 208, molecular indexer 210, compound generator 212, vectorizer 214, clusterer 216, centroid generator 218, centroid indexer 220, and/or each of the components described therein, along with any components and/or subcomponents thereof, as well as the flowcharts/flow diagrams (e.g., flowcharts 300, 400, 500, 600, and/or 700) described herein, including portions thereof, and/or further examples described herein.

Storage 820 also stores data used and/or generated by operating system 812 and application programs 814 as application data 816. Examples of application data 816 include web pages, text, images, tables, sound files, video data, and other data, which may also be sent to and/or received from one or more network servers or other devices via one or more wired or wireless networks. Storage 820 can be used to store further data including a subscriber identifier, such as an International Mobile Subscriber Identity (IMSI), and an equipment identifier, such as an International Mobile Equipment Identifier (IMEI). Such identifiers can be transmitted to a network server to identify users and equipment.

A user may enter commands and information into computing device 802 through one or more input devices 830 and may receive information from computing device 802 through one or more output devices 850. Input device(s) 830 may include one or more of touch screen 832, microphone 834, camera 836, physical keyboard 838 and/or trackball 840 and output device(s) 850 may include one or more of speaker 852 and display 854. Each of input device(s) 830 and output device(s) 850 may be integral to computing device 802 (e.g., built into a housing of computing device 802) or external to computing device 802 (e.g., communicatively coupled wired or wirelessly to computing device 802 via wired interface(s) 880 and/or wireless modem(s) 860). Further input devices 830 (not shown) can include a Natural User Interface (NUI), a pointing device (computer mouse), a joystick, a video game controller, a scanner, a touch pad, a stylus pen, a voice recognition system to receive voice input, a gesture recognition system to receive gesture input, or the like. Other possible output devices (not shown) can include piezoelectric or other haptic output devices. Some devices can serve more than one input/output function. For instance, display 854 may display information, as well as operating as touch screen 832 by receiving user commands and/or other information (e.g., by touch, finger gestures, virtual keyboard, etc.) as a user interface. Any number of each type of input device(s) 830 and output device(s) 850 may be present, including multiple microphones 834, multiple cameras 836, multiple speakers 852, and/or multiple displays 854.

One or more wireless modems 860 can be coupled to antenna(s) (not shown) of computing device 802 and can support two-way communications between processor 810 and devices external to computing device 802 through network 804, as would be understood to persons skilled in the relevant art(s). Wireless modem 860 is shown generically and can include a cellular modem 866 for communicating with one or more cellular networks, such as a GSM network for data and voice communications within a single cellular network, between cellular networks, or between the mobile device and a public switched telephone network (PSTN). Wireless modem 860 may also or alternatively include other radio-based modem types, such as a Bluetooth modem 864 (also referred to as a “Bluetooth device”) and/or Wi-Fi 862 modem (also referred to as an “wireless adaptor”). Wi-Fi modem 862 is configured to communicate with an access point or other remote Wi-Fi-capable device according to one or more of the wireless network protocols based on the IEEE (Institute of Electrical and Electronics Engineers) 802.11 family of standards, commonly used for local area networking of devices and Internet access. Bluetooth modem 864 is configured to communicate with another Bluetooth-capable device according to the Bluetooth short-range wireless technology standard(s) such as IEEE 802.15.1 and/or managed by the Bluetooth Special Interest Group (SIG).

Computing device 802 can further include power supply 882, LI receiver 884, accelerometer 886, and/or one or more wired interfaces 880. Example wired interfaces 880 include a USB port, IEEE 1394 (Fire Wire) port, a RS-232 port, an HDMI (High-Definition Multimedia Interface) port (e.g., for connection to an external display), a DisplayPort port (e.g., for connection to an external display), an audio port, an Ethernet port, and/or an Apple® Lightning® port, the purposes and functions of each of which are well known to persons skilled in the relevant art(s). Wired interface(s) 880 of computing device 802 provide for wired connections between computing device 802 and network 804, or between computing device 802 and one or more devices/peripherals when such devices/peripherals are external to computing device 802 (e.g., a pointing device, display 854, speaker 852, camera 836, physical keyboard 838, etc.). Power supply 882 is configured to supply power to each of the components of computing device 802 and may receive power from a battery internal to computing device 802, and/or from a power cord plugged into a power port of computing device 802 (e.g., a USB port, an A/C power port). LI receiver 884 may be used for location determination of computing device 802 and may include a satellite navigation receiver such as a Global Positioning System (GPS) receiver or may include other type of location determiner configured to determine location of computing device 802 based on received information (e.g., using cell tower triangulation, etc.). Accelerometer 886 may be present to determine an orientation of computing device 802.

Note that the illustrated components of computing device 802 are not required or all-inclusive, and fewer or greater numbers of components may be present as would be recognized by one skilled in the art. For example, computing device 802 may also include one or more of a gyroscope, barometer, proximity sensor, ambient light sensor, digital compass, etc. Processor 810 and memory 856 may be co-located in a same semiconductor device package, such as being included together in an integrated circuit chip, FPGA, or system-on-chip (SOC), optionally along with further components of computing device 802.

In embodiments, computing device 802 is configured to implement any of the above-described features of flowcharts herein. Computer program logic for performing any of the operations, steps, and/or functions described herein may be stored in storage 820 and executed by processor 810.

In some embodiments, server infrastructure 870 may be present in computing environment 800 and may be communicatively coupled with computing device 802 via network 804. Server infrastructure 870, when present, may be a network-accessible server set (e.g., a cloud-based environment or platform). As shown in FIG. 8, server infrastructure 870 includes clusters 872. Each of clusters 872 may comprise a group of one or more compute nodes and/or a group of one or more storage nodes. For example, as shown in FIG. 8, cluster 872 includes nodes 874. Each of nodes 874 are accessible via network 804 (e.g., in a “cloud-based” embodiment) to build, deploy, and manage applications and services. Any of nodes 874 may be a storage node that comprises a plurality of physical storage disks, SSDs, and/or other physical storage devices that are accessible via network 804 and are configured to store data associated with the applications and services managed by nodes 874. For example, as shown in FIG. 8, nodes 874 may store application data 878.

Each of nodes 874 may, as a compute node, comprise one or more server computers, server systems, and/or computing devices. For instance, a node 874 may include one or more of the components of computing device 802 disclosed herein. Each of nodes 874 may be configured to execute one or more software applications (or “applications”) and/or services and/or manage hardware resources (e.g., processors, memory, etc.), which may be utilized by users (e.g., customers) of the network-accessible server set. For example, as shown in FIG. 8, nodes 874 may operate application programs 876. In an implementation, a node of nodes 874 may operate or comprise one or more virtual machines, with each virtual machine emulating a system architecture (e.g., an operating system), in an isolated manner, upon which applications such as application programs 876 may be executed.

In an embodiment, one or more of clusters 872 may be co-located (e.g., housed in one or more nearby buildings with associated components such as backup power supplies, redundant data communications, environmental controls, etc.) to form a datacenter, or may be arranged in other manners. Accordingly, in an embodiment, one or more of clusters 872 may be a datacenter in a distributed collection of datacenters. In embodiments, exemplary computing environment 800 comprises part of a cloud-based platform such as Amazon Web Services® of Amazon Web Services, Inc. or Google Cloud Platform™ of Google LLC, although these are only examples and are not intended to be limiting.

In an embodiment, computing device 802 may access application programs 876 for execution in any manner, such as by a client application and/or a browser at computing device 802. Example browsers include Microsoft Edge® by Microsoft Corp. of Redmond, Washington, Mozilla Firefox®, by Mozilla Corp. of Mountain View, California, Safari®, by Apple Inc. of Cupertino, California, and Google® Chrome by Google LLC of Mountain View, California.

For purposes of network (e.g., cloud) backup and data security, computing device 802 may additionally and/or alternatively synchronize copies of application programs 814 and/or application data 816 to be stored at network-based server infrastructure 870 as application programs 876 and/or application data 878. For instance, operating system 812 and/or application programs 814 may include a file hosting service client, such as Microsoft® OneDrive® by Microsoft Corporation, Amazon Simple Storage Service (Amazon S3)® by Amazon Web Services, Inc., Dropbox® by Dropbox, Inc., Google Drive™ by Google LLC, etc., configured to synchronize applications and/or data stored in storage 820 at network-based server infrastructure 870.

In some embodiments, on-premises servers 892 may be present in computing environment 800 and may be communicatively coupled with computing device 802 via network 804. On-premises servers 892, when present, are hosted within an organization's infrastructure and, in many cases, physically onsite of a facility of that organization. On-premises servers 892 are controlled, administered, and maintained by IT (Information Technology) personnel of the organization or an IT partner to the organization. Application data 898 may be shared by on-premises servers 892 between computing devices of the organization, including computing device 802 (when part of an organization) through a local network of the organization, and/or through further networks accessible to the organization (including the Internet). Furthermore, on-premises servers 892 may serve applications such as application programs 896 to the computing devices of the organization, including computing device 802. Accordingly, on-premises servers 892 may include storage 894 (which includes one or more physical storage devices such as storage disks and/or SSDs) for storage of application programs 896 and application data 898 and may include one or more processors for execution of application programs 896. Still further, computing device 802 may be configured to synchronize copies of application programs 814 and/or application data 816 for backup storage at on-premises servers 892 as application programs 896 and/or application data 898.

Embodiments described herein may be implemented in one or more of computing device 802, network-based server infrastructure 870, and on-premises servers 892. For example, in some embodiments, computing device 802 may be used to implement systems, clients, or devices, or components/subcomponents thereof, disclosed elsewhere herein. In other embodiments, a combination of computing device 802, network-based server infrastructure 870, and/or on-premises servers 892 may be used to implement the systems, clients, or devices, or components/subcomponents thereof, disclosed elsewhere herein.

As used herein, the terms “computer program medium,” “computer-readable medium,” and “computer-readable storage medium,” etc., are used to refer to physical hardware media. Examples of such physical hardware media include any hard disk, optical disk, SSD, other physical hardware media such as RAMs, ROMs, flash memory, digital video disks, zip disks, MEMs (microelectronic machine) memory, nanotechnology-based storage devices, and further types of physical/tangible hardware storage media of storage 820. Such computer-readable media and/or storage media are distinguished from and non-overlapping with communication media and propagating signals (do not include communication media and propagating signals). Communication media embodies computer-readable instructions, data structures, program modules or other data in a modulated data signal such as a carrier wave. The term “modulated data signal” means a signal that has one or more of its characteristics set or changed in such a manner as to encode information in the signal. By way of example, and not limitation, communication media includes wireless media such as acoustic, RF, infrared and other wireless media, as well as wired media. Embodiments are also directed to such communication media that are separate and non-overlapping with embodiments directed to computer-readable storage media.

As noted above, computer programs and modules (including application programs 814) may be stored in storage 820. Such computer programs may also be received via wired interface(s) 880 and/or wireless modem(s) 860 over network 804. Such computer programs, when executed or loaded by an application, enable computing device 802 to implement features of embodiments discussed herein. Accordingly, such computer programs represent controllers of the computing device 802.

Embodiments are also directed to computer program products comprising computer code or instructions stored on any computer-readable medium or computer-readable storage medium. Such computer program products include the physical storage of storage 820 as well as further physical storage types.

### IV. CONCLUSION

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

In the discussion, unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of the embodiment for an application for which it is intended. Furthermore, where “based on” is used to indicate an effect being a result of an indicated cause, it is to be understood that the effect is not required to only result from the indicated cause, but that any number of possible additional causes may also contribute to the effect. Thus, as used herein, the term “based on” should be understood to be equivalent to the term “based at least on.”

While various embodiments of the present disclosure have been described above, it should be understood that they have been presented by way of example only, and not limitation. It will be understood by those skilled in the relevant art(s) that various changes in form and details may be made therein without departing from the spirit and scope of the invention as defined in the appended claims. Accordingly, the breadth and scope of the present invention should not be limited by any of the above-described exemplary embodiments, but should be defined only in accordance with the following claims and their equivalents.

What is claimed is:

1. A method for searching a chemical structure database, the method comprising:
receiving a query comprising a first molecular representation;
determining a query feature vector for the first molecular representation;
determining a first centroid feature vector with a similarity to the query feature vector that satisfies a first predetermined similarity condition, the first centroid feature vector associated with a centroid of a first group of chemical structures; and
searching a first subset of the chemical structure database to determine whether the first molecular representation is present in the first subset of the chemical structure database, the first subset of the chemical structure database comprising chemical structures of the chemical structure database associated with the first centroid feature vector.

2. The method of claim 1, wherein, responsive to determining that the first molecular representation is not present in the first subset of the chemical structure database, the method further comprises:
determining a second centroid feature vector with a similarity to the query feature vector that satisfies a second predetermined similarity condition and does not satisfy the first predetermined similarity condition, the second centroid feature vector associated with a centroid of a second group of chemical structures; and
searching a second subset of the chemical structure database to determine whether the first molecular representation is present in the second subset of the chemical structure database, the second subset of the chemical structure database comprising chemical structures of the chemical structure database associated with the second centroid feature vector.

3. The method of claim 1, wherein, responsive to determining that the first molecular representation is not present in the first subset of the chemical structure database, the method further comprises:
searching a second subset of the chemical structure database to determine whether the first molecular representation is present in the second subset of the chemical structure database, the second subset of the chemical structure database comprising at least a portion of the chemical structure database not included in the first subset of the chemical structure database.

4. The method of claim 1, wherein the first group of chemical structures comprises chemical structures that satisfy at least one of:
a Markush structure;
a chemical structure representation comprising a core molecular structure, wherein the chemical structures of the first group of chemical structures share the core molecular structure; or
a chemical structure representation comprising a molecular property, wherein the chemical structures of the first group of chemical structures share the molecular property.

5. The method of claim 1, further comprising:
receiving a first chemical structure representation;
determining a plurality of chemical structures that satisfy the first chemical structure representation;
determining a plurality of chemical structure feature vectors, each of the plurality of chemical structure feature vectors associated with one of the plurality of chemical structures;
determining the first centroid feature vector based on the plurality of chemical structure feature vectors; and
associating the first centroid feature vector with chemical structures of the chemical structure database that satisfy the first chemical structure representation.

6. The method of claim 5, wherein said determining the first centroid feature vector based on the plurality of chemical structure feature vectors comprises:
clustering the plurality of chemical structure feature vectors to determine a cluster of at least a portion of the plurality of chemical structure feature vectors; and
determining the first centroid feature vector based on the determined cluster of at least a portion of the plurality of chemical structure feature vectors.

7. The method of claim 1, wherein the first molecular representation comprises at least one of:
a machine-readable molecular representation;
a Simplified Molecular Input Line Entry System (SMILES) string;
a connection table;
an atom connectivity matrix;
a molfile;
a chemical table file;
an RGFile; or
a machine-learning representation.

8. A system for searching a chemical structure database, the system comprising:
a processor; and
a memory device that stores program code structured to cause the processor to:
receive a query comprising a first molecular representation;
determine a query feature vector for the first molecular representation;
determine a first centroid feature vector with a similarity to the query feature vector that satisfies a first predetermined similarity condition, the first centroid feature vector associated with a centroid of a first group of chemical structures; and
search a first subset of the chemical structure database to determine whether the first molecular representation is present in the first subset of the chemical structure database, the first subset of the chemical structure database comprising chemical structures of the chemical structure database associated with the first centroid feature vector.

9. The system of claim 8, wherein the program code is further structured, responsive to determining that the first molecular representation in the first subset of the chemical structure database, to cause the processor to:
determine a second centroid feature vector with a similarity to the query feature vector that satisfies a second predetermined similarity condition and does not satisfy the first predetermined similarity condition, the second centroid feature vector associated with a centroid of a second group of chemical structures; and
search a second subset of the chemical structure database to determine whether the first molecular representation is present in the second subset of the chemical structure database, the second subset of the chemical structure database comprising chemical structures of the chemical structure database associated with the second centroid feature vector.

10. The system of claim 8, wherein the program code is further structured, responsive to determining that the first molecular representation is not present in the first subset of the chemical structure database, to cause the processor to:
search a second subset of the chemical structure database to determine whether the first molecular representation is present in the second subset of the chemical structure database, the second subset of the chemical structure database comprising at least a portion of the chemical structure database not included in the first subset of the chemical structure database.

11. The system of claim 8, wherein the first group of chemical structures comprises chemical structures that satisfy at least one of:
a Markush structure;
a chemical structure representation comprising a core molecular structure, wherein the chemical structures of the first group of chemical structures share the core molecular structure; or
a chemical structure representation comprising a molecular property, wherein the chemical structures of the first group of chemical structures share the molecular property.

12. The system of claim 8, wherein the program code is structured to cause the processor to:
receive a first chemical structure representation;
determine a plurality of chemical structures that satisfy the first chemical structure representation;
determine a plurality of chemical structure feature vectors, each of the plurality of chemical structure feature vectors associated with one of the plurality of chemical structures;
determine the first centroid feature vector based on the plurality of chemical structure feature vectors; and
associate the first centroid feature vector with chemical structures of the chemical structure database that satisfy the first chemical structure representation.

13. The system of claim 8, wherein, to determine the first centroid feature vector based on the plurality of chemical structure feature vectors, the program code is structured to cause the processor to:
cluster the plurality of chemical structure feature vectors to determine a cluster of at least a portion of the plurality of chemical structure feature vectors; and
determine the first centroid feature vector based on the determined cluster.

14. The system of claim 8, wherein the first molecular representation comprises at least one of:
a machine-readable molecular representation;
a Simplified Molecular Input Line Entry System (SMILES) string;
a connection table;
an atom connectivity matrix;
a molfile;
a chemical table file;
an RGFile; or
a machine-learning representation.

15. A computer-readable storage medium comprising computer-executable instructions that, when executed by a processor, cause the processor to:
receive a query comprising a first molecular representation;
determine a query feature vector for the first molecular representation;
determine a first centroid feature vector with a similarity to the query feature vector that satisfies a first predetermined similarity condition, the first centroid feature vector associated with a centroid of a first group of chemical structures; and
search a first subset of the chemical structure database to determine whether the first molecular representation is present in the first subset of the chemical structure database, the first subset of the chemical structure database comprising chemical structures of the chemical structure database associated with the first centroid feature vector.

16. The computer-readable storage medium of claim 15, wherein the computer-executable instructions, when executed by the processor responsive to determining that the first molecular representation is not present in the first subset of the chemical structure database, further cause the processor to:
determine a second centroid feature vector with a similarity to the query feature vector that satisfies a second predetermined similarity condition and does not satisfy the first predetermined similarity condition, the second centroid feature vector associated with a centroid of a second group of chemical structures; and
search a second subset of the chemical structure database to determine whether the first molecular representation is present in the second subset of the chemical structure database, the second subset of the chemical structure database comprising chemical structures of the chemical structure database associated with the second centroid feature vector.

17. The computer-readable storage medium of claim 15, wherein the computer-executable instructions, when executed by the processor responsive to determining that the first molecular representation is not present in the first subset of the chemical structure database, further cause the processor to:
search a second subset of the chemical structure database to determine whether the first molecular representation is present in the second subset of the chemical structure database, the second subset of the chemical structure database comprising at least a portion of the chemical structure database not included in the first subset of the chemical structure database.

18. The computer-readable storage medium of claim 15, wherein the first group of chemical structures comprises chemical structures that satisfy at least one of:
a Markush structure;
a chemical structure representation comprising a core molecular structure, wherein the chemical structures of the first group of chemical structures share the core molecular structure; or
a chemical structure representation comprising a molecular property, wherein the chemical structures of the first group of chemical structures share the molecular property.

19. The computer-readable storage medium of claim 15, wherein the computer-executable instructions, when executed by the processor, cause the processor to:
receive a first chemical structure representation;
determine a plurality of chemical structures that satisfy the first chemical structure representation;
determine a plurality of chemical structure feature vectors, each of the plurality of chemical structure feature vectors associated with one of the plurality of chemical structures;
determine the first centroid feature vector based on the plurality of chemical structure feature vectors; and
associate the first centroid feature vector with chemical structures of the chemical structure database that satisfy the first chemical structure representation.

20. The computer-readable storage medium of claim 19, wherein, to determine the first centroid feature vector based on the plurality of chemical structure feature vectors, the computer-executable instructions, when executed by the processor, cause the processor to:
cluster the plurality of chemical structure feature vectors to determine a cluster of at least a portion of the plurality of chemical structure feature vectors; and
determine the first centroid feature vector based on the cluster of at least a portion of the plurality of chemical structure feature vectors.
